(19) United States
(12) Patent Application Publication
(10) Pub. No.: US 2018/0021030 A1
(43) Pub. Date: Jan. 25, 2018

(54) MORCELLATOR SPECIMEN RETRIEVAL POUCH FOR SURGICAL USE

(71) Applicant: Joseph M. FRIDLIN, Wildwood, MO (US)

(72) Inventor: Joseph M. FRIDLIN, Wildwood, MO (US)

(21) Appl. No.: 15/550,905

(22) PCT Filed: Feb. 12, 2016
(86) PCT No.: PCT/US16/17855

§ 371 Date: Aug. 14, 2017

Related U.S. Application Data
(60) Provisional application No. 62/116,054, filed on Feb. 13, 2015.

(51) Int. Cl. A61B 17/00; A61B 17/32; A61B 1/313

(52) U.S. Cl. CPC A61B 17/00234; A61B 1/313; A61B 17/320068; A61B 2017/00287; A61B 2017/320024; A61B 2018/00601

(57) ABSTRACT

Patients are benefiting from shorter hospitalization, less pain, and generally better outcomes as a result of laparoscopic surgery. The present disclosure relates to a surgical containment apparatus. More particularly, the present disclosure relates to a specimen retrieval pouch and methods for use in minimally invasive surgical procedures which is adapted to reduce cancer cell contamination through a patient's abdomen.

### CROSS-REFERENCE TO RELATED APPLICATIONS

This application claims the benefit of priority of United States provisional application No. 62/116,054 filed Feb. 13^{th}, 2015, the disclosure of which is hereby incorporated by reference as if written herein in its entirety.

### FIELD OF THE DISCLOSURE

The present disclosure relates to a surgical containment apparatus. More particularly, the present disclosure relates to a specimen retrieval pouch retrieval pouch and method for use in minimally invasive surgical procedures and more particularly, to a specimen retrieval pouch which is adapted to reduce cancer cell contamination through a patient's abdomen.

### BACKGROUND OF THE ART

Laparoscopic and endoscopic surgical procedures are minimally invasive procedures in which operations are carried out within the body by using elongated instruments inserted through small entrance openings in the body. The initial opening in the body tissue to allow passage of the endoscopic or laparoscopic instruments to the interior of the body may be a natural passageway of the body, or it can be created by a tissue piercing instrument such as a trocar. Laparoscopic and endoscopic procedures generally require that any instrumentation inserted in the body be sealed, i.e. provisions must be made to ensure that gases do not enter or exit the body through the instrument or the entrance incision so that the surgical region of the body, e.g. the peritoneum, maybe insufflated. Mechanical actuation of such instruments is for the most part constrained to the movement of the various components along a longitudinal axis with structure provided to convert longitudinal movement to lateral movement where necessary.

Because the endoscopic or laparoscopic tubes, instrumentation, and any required punctures or incisions are relatively narrow, endoscopic or laparoscopic surgery is less invasive as compared to conventional surgical procedures in which the surgeon is required to cut open large areas of body tissue. Therefore, laparoscopic or endoscopic surgery minimizes trauma to the patient and reduces patient recovery time.

Minimally invasive procedures may be used for partial or total removal of body tissue or organs from the interior of the body, e.g. nephrectomy, cholecystectomy, and other such procedures. During such procedures, it is common that a cyst, tumor, or other affected tissue or organ must be removed via the access opening in the skin, or through a cannula. Various types of entrapment devices have been disclosed to facilitate this procedure.

U.S. Pat. No. 5,037,379 discloses a surgical tissue bag for percutaneously debulking tissue by morcellation. The bag includes a layer of puncture resistant material, a layer of moisture-resistant material and a drawstring. In a disclosed method of use, the bag is placed within the body cavity, the body tissue or organ is placed within the bag, the opening of the bag is pulled through the incision in the skin leaving the distal end of the bag containing the tissue or organ within the body cavity, a morcellator is then inserted into the bag, and then the tissue or organ is debulked and suctioned out of the bag. There is increasing concern among physicians regarding the safety of a surgical procedure performed on tens of thousands of women a year in the United States to remove the entire uterus or fibroid tumors from the uterus.

The surgical procedure, known as morcellation, cuts tissue into pieces which can be pulled out of the body cavity and utilizes minimally invasive procedures, as described above. As a result, big incisions are avoided which shortens recovery time and reduces the risks of blood loss, infection, and other surgical complications.

Problems have emerged with the surgical procedure, mostly due to use of a power device. Surgeons can perform morcellation with a knife by hand however, most procedures are done with an electrical device that has a rapidly spinning blade. The surgical procedure may cause the spray of uterine tissue or fibroids around inside the abdomen. Even benign tissue, such as fibroids, can adhere and grow on organs where it does not belong, causing pain, infection and/or bowel obstruction.

### SUMMARY

These problems are overcome and a technical advantage is achieved by a specimen retrieval pouch and method for debulking tissue while containing the tissue and preventing the spread of malignant cells to healthy tissue. The specimen retrieval pouch described herein, is constructed for use with minimally invasive surgical instruments to reduce the likelihood of cancer cell contamination during a procedure.

A specimen retrieval pouch is described herein for use with electrosurgical or ultrasonic surgical devices. The specimen retrieval pouch comprises a three openings of which the surgeon can facilitate the entry and use of surgical instruments. On each lateral end of the specimen retrieval pouch there are two sleeves which can be inserted out of the patient's abdomen through access sheaths (i.e. ports), to facilitate the entry and use of surgical instruments, such as a video system or morcellator. The use of such a specimen retrieval pouch would reduce the likelihood of cancer cell contamination during a surgical procedure. The “sleeve” sections of the specimen retrieval pouch can be inserted out of the patient's abdomen through access sheaths (FIG. 3, labeled Port 2 and port 3) and a surgical instrument and video system can enter the interior of the specimen retrieval pouch which is situated in the patient's abdomen.

In one embodiment the invention describes a method of cutting tissue comprising inserting a specimen retrieval pouch through a cannula, opening specimen retrieval pouch, inserting tissue into proximal opening of the specimen retrieval pouch, closing specimen retrieval pouch around a electrosurgical or ultrasonic surgical instrument, inserting electrosurgical or ultrasonic surgical instrument and a video system into lateral ports (Port 2 or Port 3, FIG. 3) sleeves of specimen retrieval pouch, and cutting tissue with an electrosurgical or ultrasonic surgical instrument while viewing with video system.

In another embodiment, a specimen retrieval pouch is described for use during laparoscopic surgical morcellation comprising at least one lateral sleeve for inserting an electrosurgical or ultrasonic surgical instruments.

In yet another embodiment, a specimen retrieval pouch is described for use during laparoscopic surgical morcellation comprising at least two lateral sleeves for inserting an electrosurgical or ultrasonic surgical instruments and a video system.

In one embodiment, a specimen retrieval pouch is described with at least one lateral sleeve for inserting an electrosurgical or ultrasonic surgical instrument.

In a second embodiment, a specimen retrieval pouch is described with at least two lateral sleeves for inserting an electrosurgical or ultrasonic instrument and video system.

In a third embodiment, the disclosed specimen retrieval pouch reduces cancer cell contamination in a patient's abdomen during laparoscopic surgery.

A method of debulking tissue comprising inserting a specimen retrieval pouch through a cannula, opening specimen retrieval pouch, inserting tissue into proximal opening of the specimen retrieval pouch, closing specimen retrieval pouch around an electrosurgical or ultrasonic surgical instrument, inserting electrosurgical or ultrasonic surgical instrument and video system into lateral ports of specimen retrieval pouch, cutting tissue with electrosurgical or ultrasonic surgical instrument.

In another embodiment, the morcellator is placed into the specimen retrieval pouch, thus reducing bulk of the tissue or organs before withdrawal of the pouch or retrieval pouch through the incision defect, and hence improving optimal efficiency during insertion and removal of the material of the specimen retrieval pouch, and efficiently maintaining directional orientation of enclosed tissue during extracorporeal usage. In the current disclosure, the morcellator will be inserted into at least one of the lateral sleeve openings of the specimen retrieval pouch to assist in debulking of tissue or organs.

### BRIEF DESCRIPTION OF THE DRAWINGS

Embodiments of the present disclosure are described herein below with reference to the drawings wherein:

FIG. 1. This is an isometric view of a specimen retrieval pouch according to the present disclosure with a loop drawstring attached in a well-known manner about the open end of the specimen retrieval pouch (item 4).

FIG. 2. This is an isometric view of a specimen retrieval pouch according to the present disclosure. This view contains three (3) openings, items 1, 2, and 3, and is adapted for use with minimally invasive surgical and viewing instruments. Item 1 is the main open end. Item 2 and item 3 are sleeves and can be cut open or remained sealed.

FIG. 3. This is a view of standard port placement of minimally invasive surgical and viewing instruments during a laparoscopic surgical procedure. The view consists of port 1, port 2, and port 3. Each of this ports can be used to access the abdominal lumen with surgical and video instruments during a laparoscopic surgical procedure.

### DETAILED DESCRIPTION OF THE DISCLOSURE

Shorter hospitalizations, less pain, and generally better outcomes are a result of laparoscopic surgery. During these surgical procedures, large tissue masses, such as fibroid tissue masses, are traditionally excised and removed intact from the patient through the surgical incision.

Many times these masses can easily be 3 centimeters in diameter or larger. In minimally invasive surgical procedures, the operation is typically conducted using incisions of less than 1 centimeter, and often 5 millimeters or less. Thus, the trend toward the use of minimally invasive surgery has created a need to reduce large tissue masses to a size small enough to fit through an opening which may be 1 centimeter or smaller in size.

One common procedure for reducing the size of large tissue masses is generally referred to as morcellation. A surgeon performing a minimally invasive surgical procedure, will often make three small incisions in the patient's abdominal wall. A medial incision may be used for insertion of a video system, and two lateral incisions may be used for surgical instrumentation. If the incisions are small (e.g. 5 mm trocar ports), and the surgically excised organ is larger than 5 mm in diameter, the excised organ may be morcellated and removed through a trocar inserted in one of the lateral incisions.

Morcellation is facilitated by placing a specimen retrieval pouch into the abdominal cavity and opening the specimen retrieval pouch to facilitate access to the interior of the specimen retrieval pouch. Specimen retrieval pouchs which are used for morcellation may also be referred to as containment or morcellation retrieval pouches. Drawstrings for the specimen retrieval pouch opening may be either inside the abdominal cavity, or alternatively, a portion of the strings may extend through the trocar outside the patient's abdominal wall through an access sheath. The retrieval pouch opening may now be closed by pulling the drawstrings (insert number here). The surgeon may continue to pull the drawstrings out of the trocar port, bringing a portion of the specimen retrieval pouch to the outside of the patient's abdominal wall, and leaving a portion of the specimen retrieval pouch with the excised tissue and contents inside the patients abdominal cavity. The tissue in the retrieval pouch may then be morcellated to facilitate removal of the retrieval pouch and its contents through the trocar utilizing the sleeves of the disclosure described herein.

Specially designed medical instruments, which are generally referred to as morcellators, have been developed to reduce the volume of excised tissue before it is removed from the patient. Excised tissue is morcellated (i.e. debulked), collected and removed from the patient's body through, for example, a surgical trocar or directly through one of the surgical incision.

Mechanical morcellators cut tissue using, for example, sharp end-effectors such as rotating blades. Electrosurgical and ultrasonic morcellators use energy to morcellate tissue. In order to prevent morcellated tissue from spreading to other parts of the body during and after the morcellation procedure, the excised tissue is, in most cases, placed in a specimen retrieval pouch described herein, prior to being morcellated.

In another embodiment described herein, a surgeon can insert a surgical device through the specimen retrieval pouch as described herein, and pull an organ, i.e. lung lobe, into the retrieval pouch rather than trying to insert an organ into the bag from the abdominal cavity. This latter surgical procedure frequently fails when the surgeon attempts to release surgical instruments from the abdominal cavity.

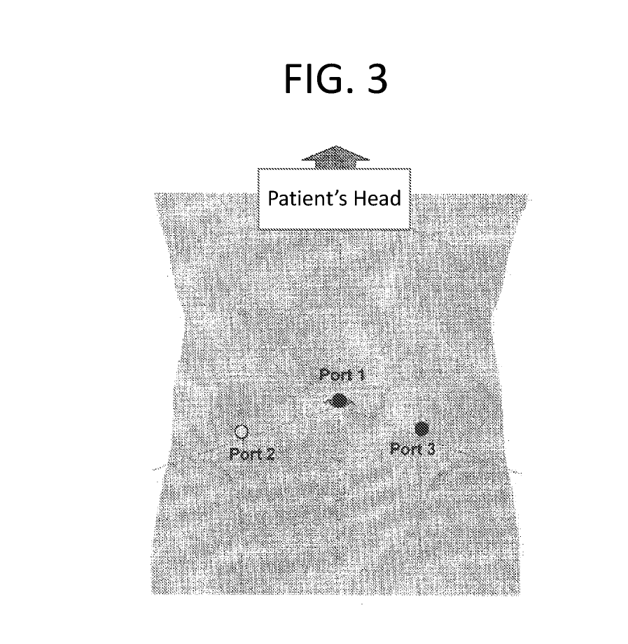

In yet another embodiment described herein, the specimen retrieval pouch comprises three openings, two of which are sleeves, which can be inserted through access sheaths (Port 2 and Port 3 of FIG. 3). The surgeon may cut the sleeves of the specimen retrieval pouch outside of the body allowing access at three different triangulated points (FIG. 3) to manipulate the excised organ or specimen with the specimen retrieval pouch while preventing spread and contamination of the abdominal cavity with malignant tissue.

In another embodiment the specimen is inside the retrieval pouch and the main open end is outside a port by pulling the draw string. A surgeon can reach through any of the other two trocars and grab either of the sleeves of the specimen retrieval pouch. The sleeves can be pulled out of the access ports, in addition to pulling those trocars out of the patient, and cutting the tips off of either or both sleeves, and then inserting the trocars (ports) into the cut sleeves and back into the patient. With the main open end and the sleeves of the specimen retrieval pouch extended to the outside of the patient, there are three port entries into the specimen retrieval pouch inside the patient abdomen and to the specimen. This allows for manipulation and cutting of the specimen with three different access points with the barrier of protection from all points extending all the way outside the patient and eliminating the risk of contamination. In another embodiment, the surgeon can insufflate inside the specimen retrieval pouch rather than inside the patient. This will reduce exposure of CO2 to the patient and ultimately assist in recovery time, infection, and other surgical complications.

As used herein with reference to the present disclosure, the terms “laparoscopic” and “endoscopic” are interchangeable and refer to instruments having a relatively narrow operating portion for insertion into a cannula or a small incision in the skin, or to a surgical procedure in which such instruments are employed. Use herein of the term “laparoscopic” should not be construed to exclude “endoscopic” and use herein of the term “endoscopic” should not be construed to exclude “laparoscopic.”

As used herein, laparoscopic instruments, include, but are not limited to any instrument used for cutting, grasping or stitching such as a crocodile grasper, cartier, debakey, tennaculum, needle driver, or scissors.

As used herein, a trocar is a pen-shaped instrument with a sharp triangular point at one end, typically used inside a hollow tube, known as a cannula or sleeve, to create an opening into the body through which the sleeve may be introduced, to provide an access port during surgery.

To the contrary, it is believed that the present disclosure may find use in any procedure where access to the interior of the body is limited to a relatively small incision, with or without the use of a cannula, including, but not limited to, laparoscopic procedures. Uterine fibroids are noncancerous growths that develop from the muscular tissue of the uterus. Most women will develop uterine fibroids (also called leiomyomas) at some point in their lives, although most cause no symptoms. In some cases, however, fibroids can cause symptoms, including heavy or prolonged menstrual bleeding, pelvic pressure or pain, and/or frequent urination, requiring medical or surgical therapy.

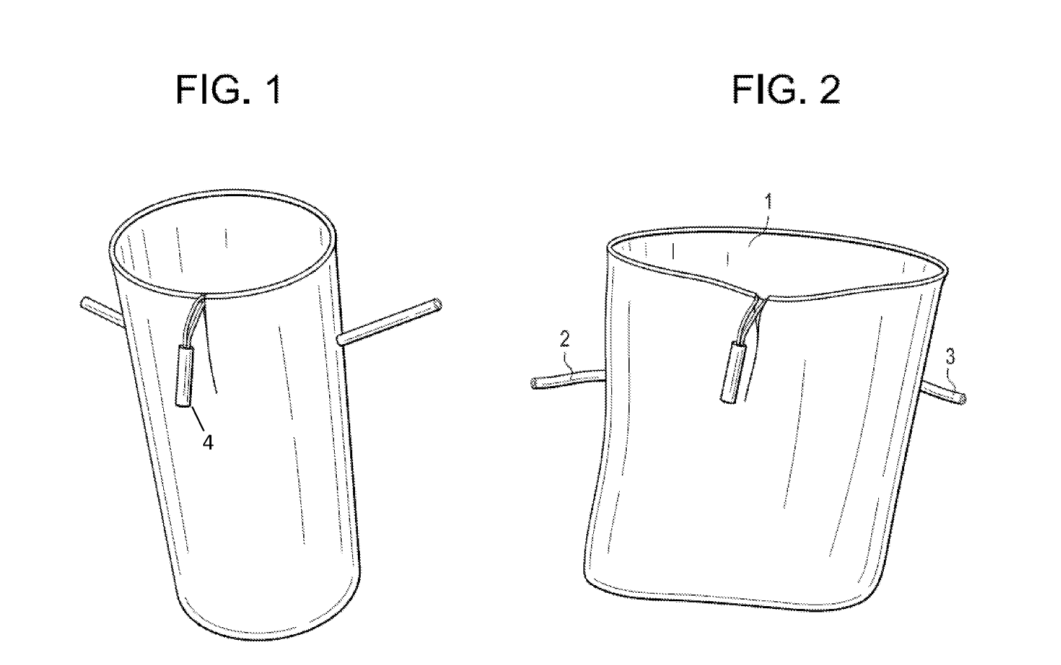

Depicted in FIG. 1 is a surgical specimen retrieval pouch for debulking tissue contained therein and preventing the spread of malignant cells to healthy tissue within a surgical site. The specimen retrieval pouch is comprised of a flexible and foldable material for insertion into a body cavity through an access sheath inserted into the body cavity for a minimally invasive surgical procedure as described above. The flexible material of the specimen retrieval pouch maintains a gas-tight seal while traversing the percutaneous puncture site. The flexible material also compliantly plugs the puncture site, thus preventing any significant loss of body cavity insufflating gas.

The tissue specimen retrieval pouch comprises a puncture-resistant material such as rip stop nylon fabric, Resinado, Sinylon, Osmo Elite, Sport Canvas, or Seattle Fabric in a woven form for resisting penetration by a surgical morcellating instrument. This material will also prevent the transmission of fluid or malignant tissue cells to healthy tissue within the surgical site. The tissue specimen retrieval pouch also includes closure means such as a loop drawstring attached in a well-known manner about the open end of the specimen retrieval pouch and has a portion extending from the open end and having a length extendable through an access sheath during the surgical procedure for drawing the open end of the specimen retrieval pouch closed and pulling the closed open end of the specimen retrieval pouch from the cavity and through the puncture site.

Many women choose to undergo laparoscopic hysterectomy or myomectomy because these procedures are associated with benefits such as a shorter post-operative recovery time and a reduced risk of infection compared to abdominal hysterectomy and myomectomy. Many of these laparoscopic procedures are performed using a power morcellator.

When used for hysterectomy or myomectomy in women with uterine fibroids, laparoscopic power morcellation poses a risk of spreading unsuspected cancerous tissue, notably uterine sarcomas, beyond the uterus. Specimen retrieval pouches are, therefore, designed to hold excised tissue without spilling tissue, or tissue components, into the abdominal cavity during power morcellation.

However, limitations exist in the prior art. Blades on power morcellators have cause tears or cuts into the specimen retrieval pouch causing contents to spill out of the specimen retrieval pouch and into patient's abdomen. This problem can be alleviated using a specimen retrieval pouch with at least one additional opening to facilitate the entry and use of surgical instruments, such as a video system and morcellator. The use of such a device will assist a surgeon's ability to view tissue during morcellation procedure and continue to use the triangular procedure of laparoscopic surgery.

What is claimed is:

1. A minimally invasive specimen retrieval pouch for debulking tissue comprising an open end with a drawstring attached and one or more sleeves positioned on lateral ends of the specimen retrieval pouch wherein said drawstring has a length extendable through a sheath in Port 1 of an abdominal cavity during a surgical procedure for drawing said open end closed and the said one or more sleeves are extendable through Port 2 and Port 3.

2. The specimen retrieval pouch of claim 1, wherein in the one or more sleeves can be inserted through access sheaths in the abdominal cavity.

3. The specimen retrieval pouch of claim 1, wherein in the one or more sleeves are used for insertion of surgical equipment and a camera system.

4. The specimen retrieval pouch of claim 2, wherein specimen retrieval pouch is made of puncture-resistant material.

5. The specimen retrieval pouch of claim 3, wherein the specimen retrieval pouch is made of stop nylon fabric, Resinado, Sinylon, Osmo Elite, Sport Canvas, or Seattle Fabric.

6. The specimen retrieval pouch claim 1, wherein the specimen retrieval pouch reduces cancer cell contamination in a patient's abdomen.

7. A method of debulking tissue comprising inserting a specimen retrieval pouch through a cannula, opening the specimen retrieval pouch end with a drawstring attached, pulling tissue into specimen retrieval pouch, and cutting tissue with electrosurgical or ultrasonic surgical instrument.

8. A method of cutting tissue comprising the steps of:
a. inserting a specimen retrieval pouch through a cannula;
b. opening specimen retrieval pouch;
c. inserting tissue into proximal opening of the specimen retrieval pouch;
d. inserting electrosurgical or ultrasonic instrument and video system into lateral ports of specimen retrieval pouch;
e. cutting tissue with an electrosurgical or ultrasonic instrument.

9. A specimen retrieval pouch for use during laparoscopic surgical morcellation comprising at least one sleeve for inserting an electrosurgical or ultrasonic instrument.

10. A specimen retrieval pouch for use during laparoscopic surgical morcellation comprising at least two lateral sleeves for inserting an electrosurgical or ultrasonic instrument and a video system.

11. A method according to claim 7 wherein the specimen retrieval pouch comprising at least one lateral sleeve for inserting an electrosurgical and ultrasonics instrument.

12. A method according to claim 7 wherein the specimen retrieval pouch comprising at least two lateral ports for inserting an electrosurgical and ultrasonic instrument and video system.

13. A method of debulking tissue comprising the steps of:
a. inserting a specimen retrieval pouch through a cannula;
b. opening specimen retrieval pouch;
c. pushing tissue into proximal opening of the specimen retrieval pouch;
d. inserting electrosurgical and ultrasonic instrument and video system into lateral sleeves pushing specimen retrieval pouch;
e. cutting tissue with an electrosurgical or ultrasonic instrument.

14. A method according to claim 13 wherein the specimen retrieval pouch comprising at least one lateral sleeve for inserting an electrosurgical and ultrasonic instrument.

15. A method according to claim 13 wherein the specimen retrieval pouch comprising at least two lateral sleeves for inserting an electrosurgical or ultrasonic instrument and video system.
